(19) United States
(12) Patent Application Publication
(10) Pub. No.: US 2014/0213981 A1
(43) Pub. Date: Jul. 31, 2014

(54) BANDAGE BAG

(71) Applicants: Joseph BOCKOL, Calabasas, CA (US); Marla Anne SCHRAM, Calabasas, CA (US); John Jeffrey RUSH, Potomac, MD (US)

(72) Inventors: Joseph BOCKOL, Calabasas, CA (US); Marla Anne SCHRAM, Calabasas, CA (US); John Jeffrey RUSH, Potomac, MD (US)

(21) Appl. No.: 13/753,168

(22) Filed: Jan. 29, 2013

(51) Int. Cl. A61M 25/02

(52) U.S. Cl. CPC A61M 25/02
USPC 604/180

(57) ABSTRACT

A bandage bag configure to provide the dual functions of holding a medical apparatus to a patient for daily wear and of serving as a waterproof bandage, especially for a catheter and its insertion site.

### FIELD OF THE INVENTION

This application relates to a device for securing to a patient a medical apparatus that protrudes, in part, beyond the skin. The device carries or encloses the apparatus and also serves as a waterproof and/or protective bandage or dressing.

### BACKGROUND

A central venous catheter, or vascular access device, is a long, thin, flexible tube used to deliver to a patient medications, fluids, nutrients, or other materials over a long period of time, usually several weeks or more.

The catheter can be placed in a large vein near the patient's heart, such as the superior vena cava, or another large vein in the arm, neck, or chest. The catheter provides several medical advantages. It can be left in place longer than an intravenous catheter, which is located in a vein near the skin surface. It can be utilized to quickly deliver medications that affect the heart, especially if an immediate response is desired. It can also be used to measure blood pressure in the superior vena cava, which can help diagnose certain heart problems.

A peripherally inserted central catheter (PICC) line is a central venous catheter which is inserted into a peripheral vein, for example in the arm of a patient, and then threaded through progressively larger veins into the superior vena cava. PICC lines are the most popular way to administer outpatient therapy by a central vein.

The handling of PICC lines in daily life can be difficult. Approximately seven inches of tubing and port will usually hang from the patient's arm and must be cared for.

In addition, the catheter insertion site (where the PICC line is inserted), tubing, and port must be kept dry to prevent infection. Caring for the insertion site can be difficult. Nurses are trained to dress the insertion site with an antiseptic treatment and then place a clear protective adhesive over the area. On a periodic basis, the dressing is removed and the area is inspected, cleaned, and redressed. Removal of the adhesive can be painful. Finally, the PICC line is unsightly. Patients are discouraged from wearing long-sleeves, thus exposing a noticeable medical condition.

Some methods have been used to combat only problems of bathing. One remedy is to use a tubular sleeve and seal it at both ends, leaving the insertion site inside the sleeve. For example, U.S. Pat. No. 6,276,364 discloses a waterproof sleeve with an elastomeric band at each end to seal the sleeve. In another variation, one end of the sleeve is permanently sealed. For example, U.S. Pat. Nos. 5,605,534 and 5,720,713 each comprise a large bag which encases the entire arm and is closed off with a flexible band at the open end. Such solutions are not optimal. These remedies allow for bathing only. In the case of the large bag-type method, one hand is necessarily covered up by it, which increases the difficulty of doing many things. None of these methods allow for an elegant solution to the problem of the daily dressing of a PICC line. In addition, these remedies are cumbersome and bulky. They are not bandages in any sense; they are only water-resistant “rain coats” for the arm. They are not designed for daily wear.

There remains a need for a device which has the dual functions of holding medical apparatus (such as tubing and port) and of being able to serve as a protective bandage or dressing. Such a device should preferably restrict the patient's range of motion as little as possible, be light, and be easy to apply by oneself.

### SUMMARY

A first object is to provide an improved bandage bag device.

A second object is to provide a bandage bag device comprising or consisting of a bag and a flap, the flap being connected to the bag.

A third object is to provide a bandage bag device comprising or consisting of a bag and a flap, the flap being connected to the bag by a hinge.

A fourth object is to provide a bandage bag device comprising or consisting of a bag and a flap, the flap being connected to the bag by a live hinge.

A fifth object is to provide a bandage bag device comprising or consisting of a layer of flexible material.

A sixth object is to provide a bandage bag comprising or consisting of a layer of flexible material, the layer comprising zones of different thickness.

A seventh object is to provide a bandage bag comprising a layer of flexible material comprising or consisting of a pair of thicker zones connected together by a thinner zone defining a foldable hinge between the thicker zones.

An eighth object is to provide a bandage bag comprising or consisting of a three dimensional layer of flexible material.

Disclosed herein, in various embodiments, are bandage bags which have a dual function of holding or enclosing a medical apparatus protruding beyond the skin (i.e. tubing, ports, catheters, etc.) and serving as a bandage or dressing for daily wear.

In one embodiment, the bandage bag comprises a rectangular or square enclosure or receptacle bag and two adhesive layers. The bag is open along a superior edge for placing medical apparatus therein. The first adhesive layer is placed on the anterior side of the bag along three edges: a first lateral edge, an inferior edge, and a second lateral edge. The second adhesive layer is placed on the posterior face of the bag along the superior edge.

In additional embodiments, the bandage bag further comprises at least one wing which is located on the posterior face along the superior edge and is attached to a lateral edge such that the wing can be pivoted to extend laterally from the bag or to cover at least a portion of the second adhesive layer. In more specific embodiments, the at least one wing has a first side which contacts at least a portion of the second adhesive layer and has a third adhesive layer disposed on the first side.

In further embodiments, the bandage bag has a total of two wings. One wing is attached to the first lateral edge and the other wing is attached to the second lateral edge.

In still further embodiments, the bandage bag further comprises a removable backing or release layer shaped to cover the first adhesive layer.

In other embodiments, further materials may be disposed on the anterior face of the bandage bag. These materials aid in treating and dressing the catheter insertion site.

In still additional embodiments, alternative configurations, such as triangular, circular, elliptical, and other suitable shapes, can be utilized. The bandage bag may also be sized or shaped to meet or conform to the patient's specific body configurations. In such configurations, the adhesive layer(s) are preferably placed on the outer or peripheral edges of the bandage bag.

Methods for using the bandage bag to contain medical apparatus and to form a protective and/or waterproof bandage or dressing over a catheter insertion site are also provided. The bandage bag is attached near the catheter insertion site with the second adhesive layer. Medical apparatus are placed inside the bag. The bag is then folded across a horizontal axis such that the catheter insertion site is covered by the anterior face of the bandage bag. The protective and/or waterproof bandage is then formed by attaching the bandage bag with the first adhesive layer such that it surrounds the insertion site.

These and other non-limiting features or characteristics of the present disclosure will be further described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is an anterior elevational view of an exemplary embodiment of the bandage bag.

FIG. 2 is a posterior elevational view of an exemplary embodiment of the bandage bag wherein the bandage bag has two wings.

FIG. 3 is a perspective view showing one use of the bandage bag.

FIG. 4 is a perspective view showing the bandage bag when folded over as a waterproof bandage.

FIG. 5 is a posterior elevational view of an exemplary embodiment of the bandage bag wherein the bandage bag has one wing.

FIG. 6 is an anterior elevational view of an exemplary embodiment of the bandage bag wherein the opening of the bandage bag is sealed on the ends of the superior edge.

FIG. 7 is an anterior elevational view of an exemplary embodiment of the bandage bag wherein an impregnated material is present on the anterior face of the bandage bag.

FIG. 8 is an anterior elevational view of another exemplary embodiment of the bandage bag.

FIG. 9 is a side elevational view of the embodiment shown in FIG. 8.

FIG. 10 is a posterior elevational view of the embodiment shown in FIGS. 8 and 9.

FIG. 11 is an anterior elevational view of the embodiment of the bandage bag shown in FIG. 8.

FIG. 12 is a partial broken away enlarged detailed view of the area of a portion of the bandage bag, as indicated in FIG. 9.

FIG. 13 is a partial broken away enlarged detailed cross-sectional view of the bandage bag as indicated in FIG. 8.

FIG. 14 is an anterior elevational view of a further exemplary embodiment of the bandage bag.

FIG. 15 is a side elevational view of the embodiment of the bandage bag shown in FIG. 14.

FIG. 16 is an anterior elevational view of an even further exemplary embodiment of the bandage bag.

FIG. 17 is an anterior elevational view of a further exemplary embodiment of the bandage bag.

FIG. 18 is a posterior elevational view of an even further exemplary embodiment of the bandage bag.

### DETAILED DESCRIPTION

The exemplary embodiments of this application are more particularly described below with reference to the drawings. Although specific terms are used in the following description for clarity, these terms are intended to refer only to the particular structure of the various embodiments selected for illustration in the drawings and not to define or limit the scope of the application. The same reference numerals are used to identify the same structure in different Figures. The structures in the Figures are not drawn according to their relative proportions and the drawings should not be interpreted as limiting the application in size, relative size, or location.

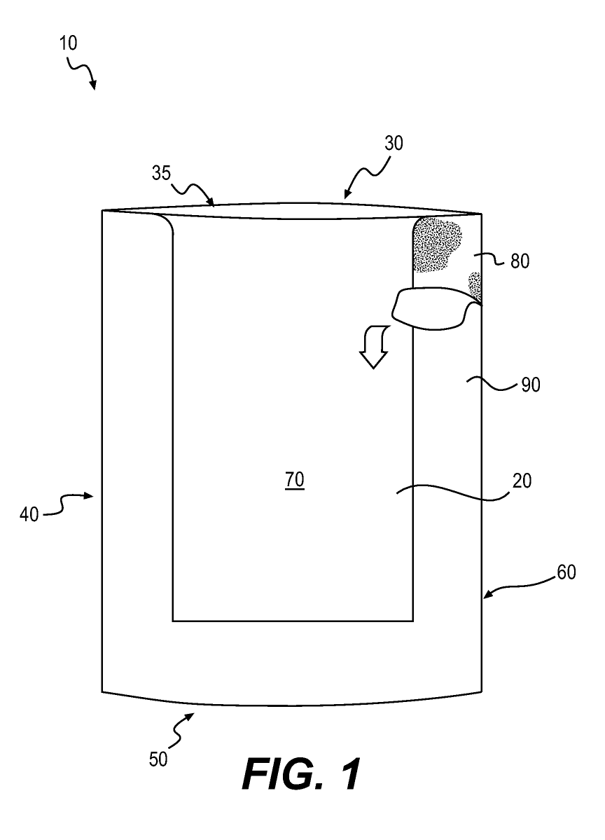

FIG. 1 is an anterior, or front, view of an exemplary embodiment of the bandage bag. The bandage bag 10 comprises an enclosure bag 20. The enclosure bag 20 is open along a superior edge 30 and can be accessed through an opening 35 to place items, such as medical apparatus, inside the enclosure bag 20. The enclosure bag 20 has a first lateral edge 40, an inferior edge 50, and a second lateral edge 60. The anterior face 70 of the enclosure bag 20 is visible. A first adhesive layer 80 is disposed along the first lateral edge 40, inferior edge 50, and second lateral edge 60. Note that the adhesive 80 does not completely cover the anterior face 70, but only its edges. A removable backing 90 is shaped to cover the first adhesive layer 80 for ease and convenience in handling. The backing 90 is removed when the bandage bag 10 is used.

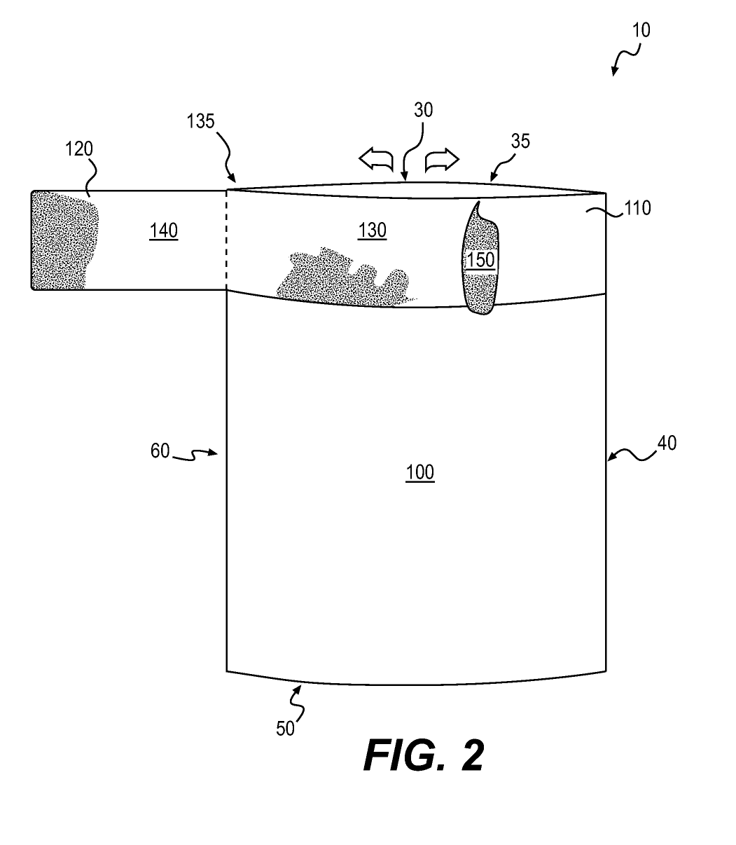

FIG. 2 is a posterior, or back, view of an exemplary embodiment of the bandage bag. A second adhesive layer 130 is disposed along the superior edge of the posterior face 100. Again, note the posterior face 100 is not completely covered by the adhesive layer 130. In the depicted embodiment, the bandage bag 10 has two wings: a first wing 110 and a second wing 120. The wings are located on the posterior face 100 along the superior edge 30. Each wing is attached to a lateral edge such that the wing can be pivoted to extend laterally from the enclosure bag 20. For example, the second wing 120 is attached to the second lateral edge 60 at the attachment point 135 and extends laterally. A third adhesive layer 140 is disposed upon the second wing 120. The first wing 110 is shown covering approximately one-half of the second adhesive layer 130. Part of the third adhesive layer 150 on the first wing 110 is visible. The wings are both extended laterally, from the enclosure bag 20, and since they each have a third adhesive layer, can be secured to the arm.

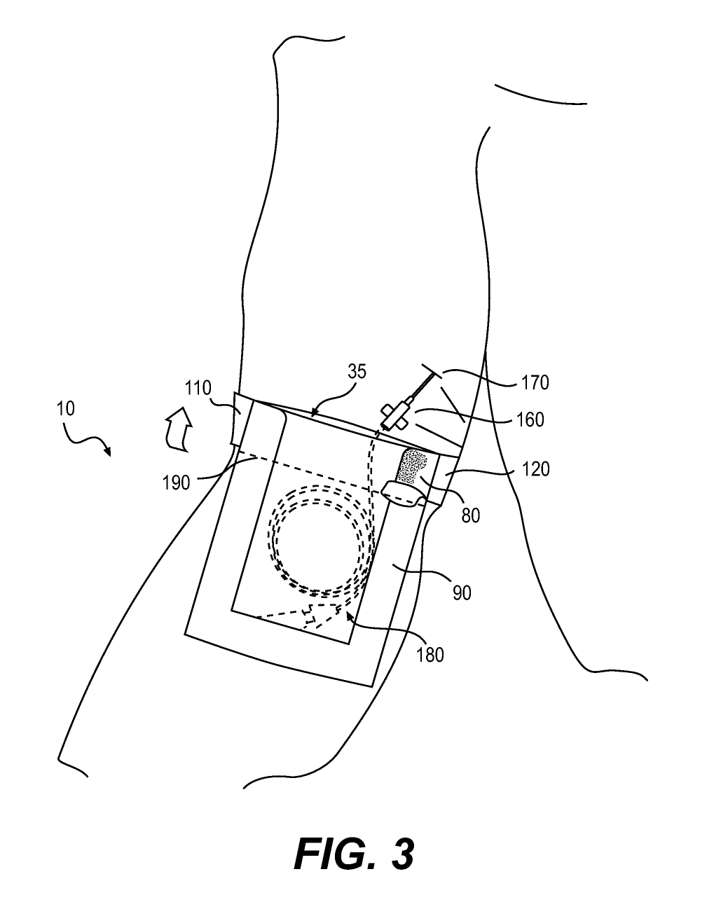

FIG. 3 is a diagram showing one use of the bandage bag. The diagram depicts the arm of a patient with a PICC inserted. The catheter 160 and the catheter insertion site 170 are visible on the arm. The anterior face of the bandage bag 10, with the first adhesive layer 80 and the removable backing 90, is also visible. Here, the bandage bag 10 is placed above the antecubital fossa (the fold of the elbow) and near the catheter insertion site 170. The two wings 110 and 120 have been extended laterally to expose the second adhesive layer and the third adhesive layer of each wing; these adhesive layers attach the bandage bag to the arm of the patient. The medical apparatus 180 (tubing and port in this diagram) have been inserted into the bandage bag 10 through the bag opening 35. The bandage bag is then folded across a horizontal axis 190 such that the catheter insertion site is covered by the anterior face of the bandage bag and the first adhesive layer 80 faces the arm. A waterproof bandage or dressing is formed by attaching the bandage bag 10 to the arm with the first adhesive layer 80. A pocket will be formed on three sides by the first adhesive layer 80 and on the fourth side by the fold along the horizontal axis 190. The catheter insertion site 170 is located within the pocket created by the fold. Note that the horizontal axis 190 may be located almost anywhere along the length of the bandage bag as long as the bag opening 35 is closed after the bandage bag has been folded. In practice, however, the horizontal axis 190 will generally be near the superior edge of the bandage bag.

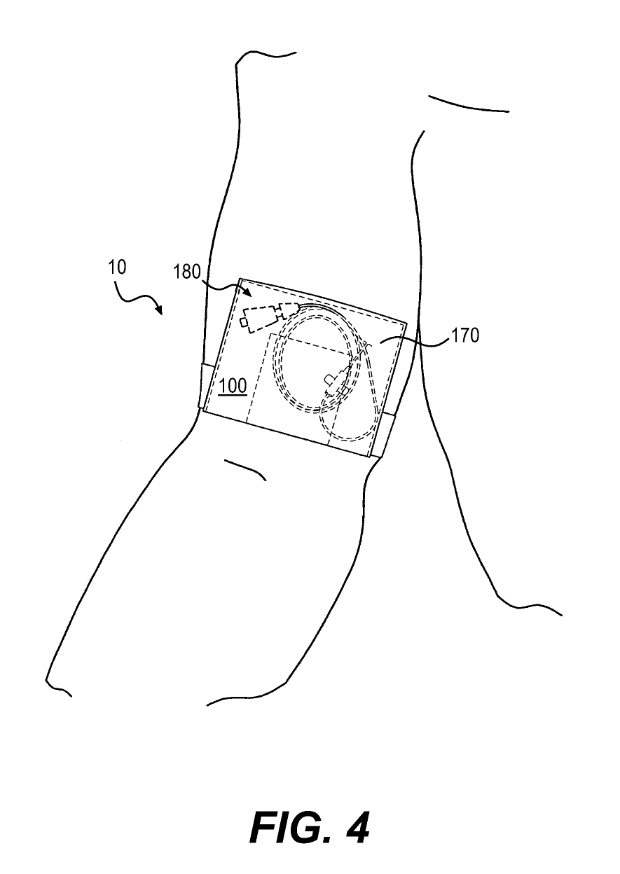

FIG. 4 shows the bandage bag after it has been folded over as a waterproof bandage. The posterior face 100 of the bandage bag 10 is now visible. The medical apparatus 180 and the catheter insertion site 170 are now segregated from the outside environment by the water-resistant seal. In particular, the catheter insertion site 170 is protected from moisture and other dirt or debris. This decreases the chance of infection, promoting faster healing and patient well-being. It also allows the patient to bathe and shower without hassle or worry. The bandage bag allows the tubing and port to be contained securely, eliminating the need for daily maintenance. The waterproof bandage is also easier to apply and covers a smaller portion of the arm than other larger non-bandage devices that are solely intended for use while bathing. Note that the elbow in particular is not covered, allowing the patient to have full range of motion. The insertion site is discreetly covered, so others cannot see the wound. The bandage bag is also thin and light, which allows the wearing of long-sleeve clothing.

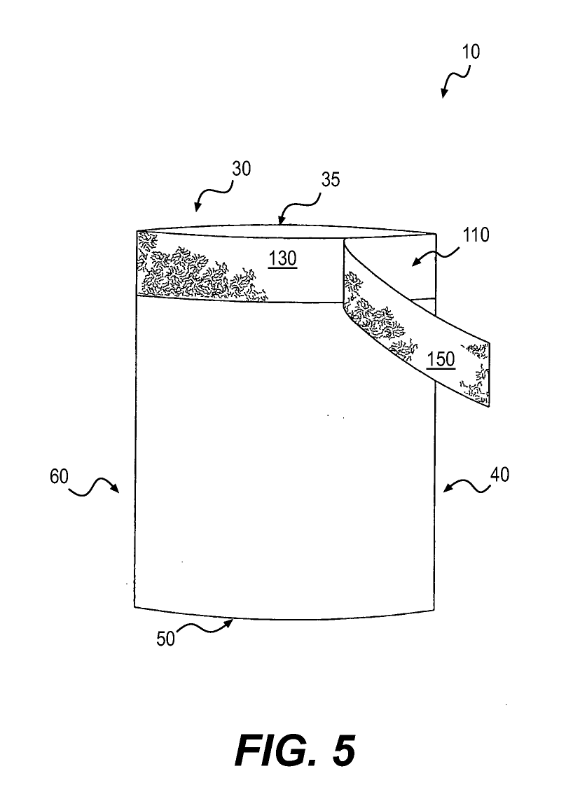

FIG. 5 is a posterior view of another exemplary embodiment of the bandage bag. This embodiment differs from that depicted in FIG. 2 in that the bandage bag 10 has only one wing 110. The wing 110 entirely covers the second adhesive layer 130 when folded against the superior edge 30 of the bandage bag. Here, the wing 110 is again attached to the first lateral edge 40. A third adhesive layer 150 is disposed on one side of the wing 110.

The enclosure bag portion 20 of the bandage bag can be made from a flexible, waterproof material. Such materials may include cellophane-like materials, including, but not limited to, plastic, polymer, latex, or rubber. It may have any dimensions desired. In specific embodiments, the bag is square or rectangular (prior to being folded). In one specific embodiment, the bag has dimensions of about 8 cm in length and about 8 cm in width. The bag opening 35 generally runs along the entirety of the superior edge 30. However, this is not required and the opening may be less than the entirety of the superior edge. For example, the bag opening may run only within the middle of the superior edge so that the superior edge is sealed at its ends (near the lateral edges) in some embodiments. When the bandage bag is folded over, the opening 35 will then be wholly contained within the waterproof pocket.

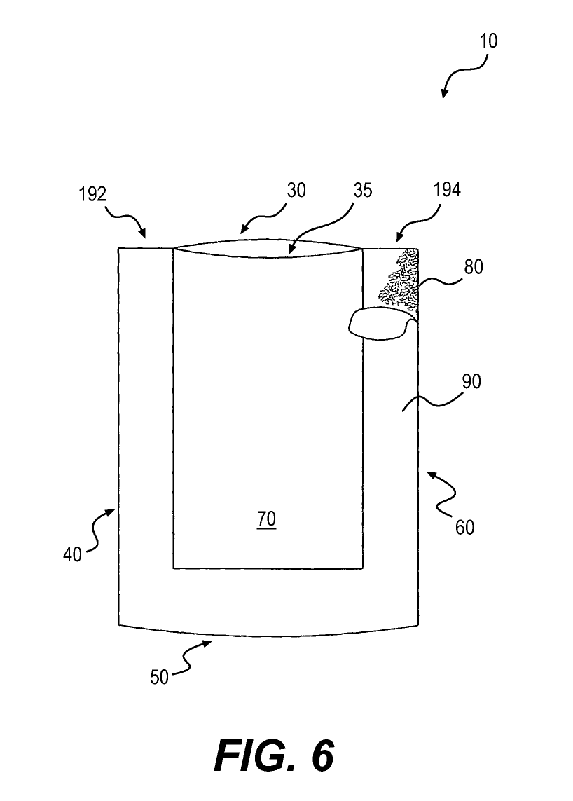

FIG. 6 shows such an embodiment. In this anterior view of the bandage bag 10, the bag opening 35 does not constitute the entirety of the superior edge. Instead, the superior edge is sealed at a first superior end 192 and a second superior end 194. Each superior end adjoins a lateral edge 40 or 60. The bag opening 35 runs only within the middle of the superior edge, from the first superior end 192 to the second superior end 194.

Generally, the adhesive used in the first, second, and third adhesive layers should be a medical-grade adhesive. The adhesive should also be water-resistant and non-irritating to the skin.

In embodiments where the bandage bag does not have wings, a removable backing is provided to cover both the first and second adhesive layers. In exemplary embodiments, however, the bandage bag has at least one wing. The at least one wing is located on the posterior face along the superior edge and is attached to a lateral edge such that the wing can be pivoted to extend laterally from the bag or to cover at least a portion of the second adhesive layer. In the preferred embodiment, the bandage bag has a total of two wings, wherein one wing is attached to the first lateral edge and the other wing is attached to the second lateral edge.

As can be seen in FIG. 1, the first adhesive layer 80 does not cover the whole of the anterior face 70. If desired, other materials can be disposed on the anterior face of the bandage bag. Such materials would, once the bandage bag has been folded, be contained within the pocket containing the catheter insertion site 170. For example, a water absorbent material could be placed there to soak up any moisture which might form inside the waterproof seal (for example, from condensation or sweat). Similarly, an antibiotic and/or antifungal impregnated material could be placed there to prevent any bacterial or fungal growth in or around the catheter insertion site.

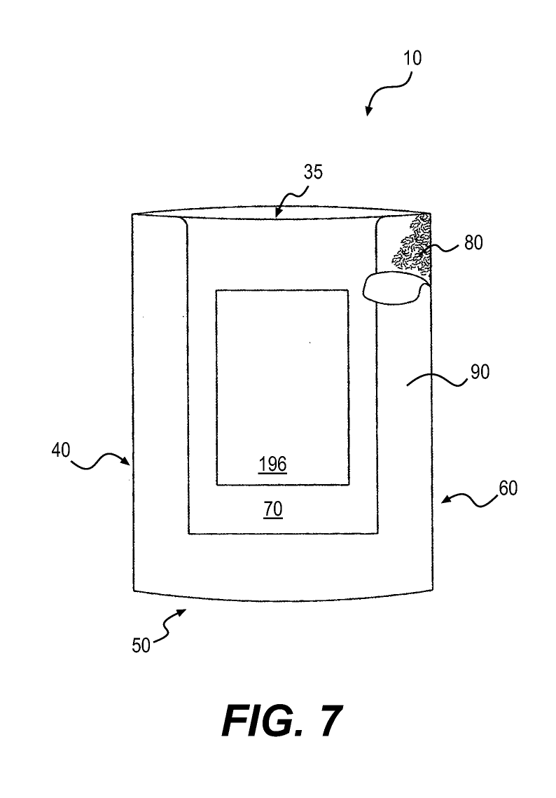

FIG. 7 is an anterior view of such an exemplary embodiment. Here, a material pad 196 is present on the anterior face 70 of the bandage bag 10. This pad could be impregnated with an antibiotic and/or antifungal. It could also be water-absorbent. Although a material pad is depicted, a pad is not required to place such materials on the anterior face and is simply used here for clarity. Antibiotic or antifungal could be applied to the anterior face in a cream or paste, for example.

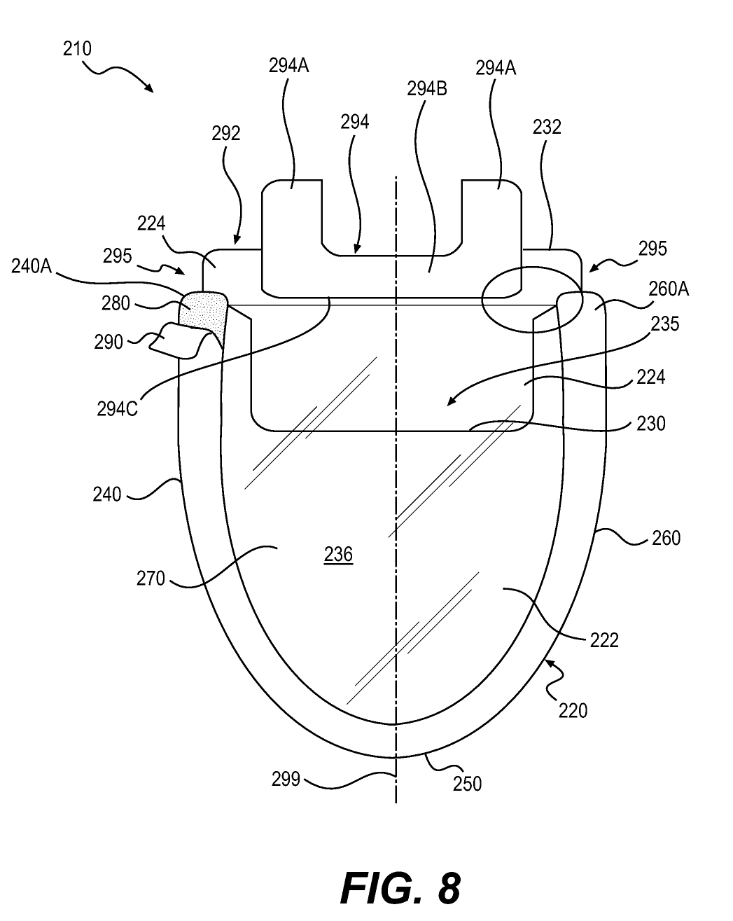

FIG. 8 is an anterior, or front, view of another exemplary embodiment of the bandage bag 194, in a condition prior to being applied to a patient. The bandage bag 194 comprises an enclosure bag 196. The enclosure bag 196 includes a superior edge 230 on the anterior wall 222 of the enclosure bag 196 and a superior edge 232 on the posterior wall 224 of the enclosure bag 196 defining an opening 235 into a pocket 236. The enclosure bag 196 can be accessed through the opening 235 to place items, such as medical apparatus, inside the enclosure bag 196. The enclosure bag 196 has a first lateral edge 240, an inferior edge 250, and a second lateral edge 260. The anterior face 270 of the enclosure bag 196 is visible. A first adhesive layer 280 is disposed along the first lateral edge 240, inferior edge 250, and second lateral edge 260. Note that the adhesive 280 does not completely cover the anterior face 270, but only its edges. A removable backing 290 is shaped to cover the first adhesive layer 280 for ease and convenience in handling. The backing 290 is removed when the bandage bag 194 is used.

A flap 292 is provided at an upper portion of the enclosure bag 196. Specifically, the flap 292 is defined by an upper wall portion 224a of the posterior wall 224 of the enclosure bag 196. The flap 292 comprises a U-shaped member 294 comprising two spaced apart wings 294A, 294A connected together by a lower member portion 294B. The wings 294A, 294A are oriented parallel to a longitudinal centerline 299 of the bandage bag 194.

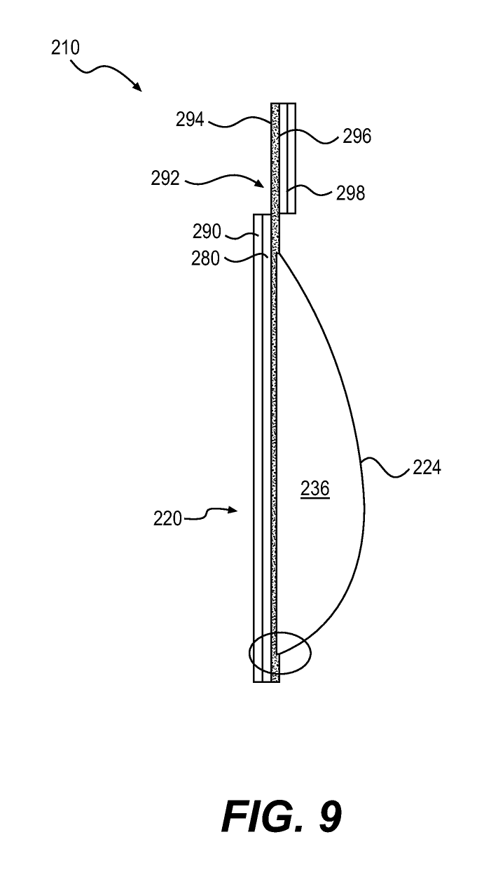

FIG. 9 is a side elevational view of the bandage bag 194 shown in FIG. 8. The posterior wall 224 of the enclosure bag 196 is curved outwardly to define the pocket 236 to place items, such as medical apparatus, inside the enclosure bag 196. The posterior side of the U-shaped member 294 is provided with an adhesive layer 296 and a removable backing 298.

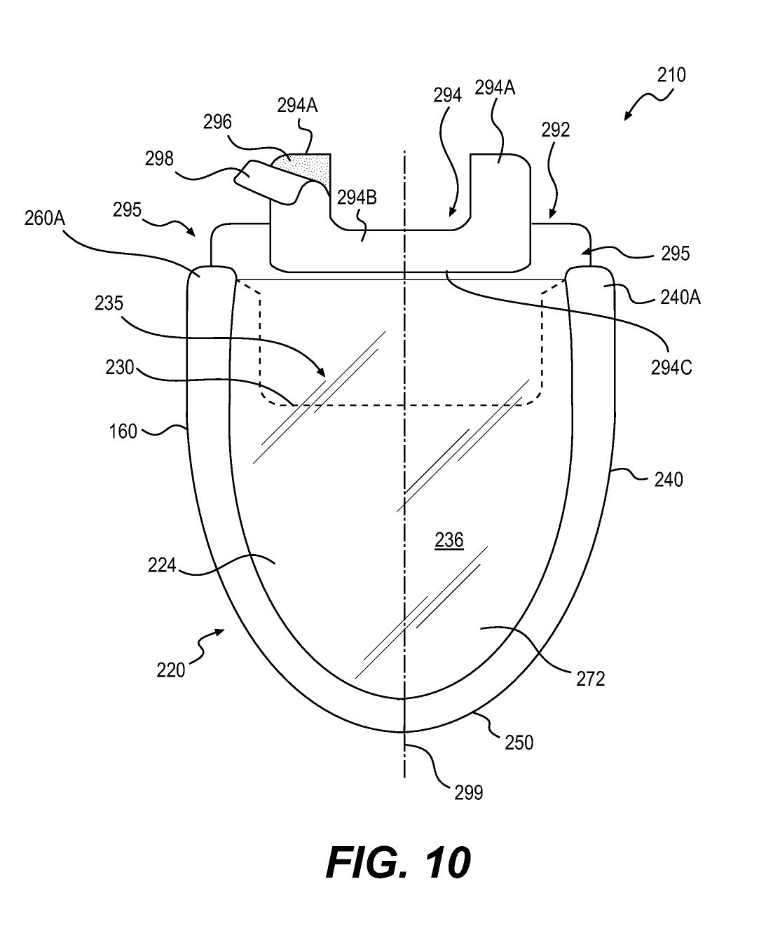

FIG. 10 is a posterior, or back, view of the bandage bag 194 shown in FIG. 8, again in an open position (i.e. unfolded). A second adhesive layer 296 is disposed along the posterior side of the U-shaped member 294.

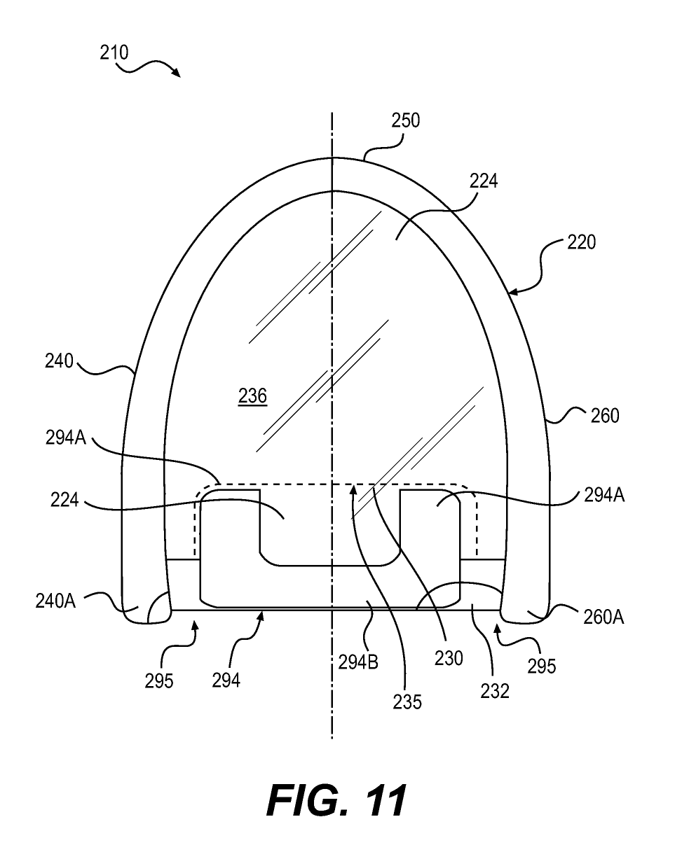

FIG. 11 is an anterior, or front, view of the bandage bag 194 shown in FIG. 8 in a closed position (i.e. folded). A second adhesive layer 296 is disposed along the posterior side of the U-shaped member 294.

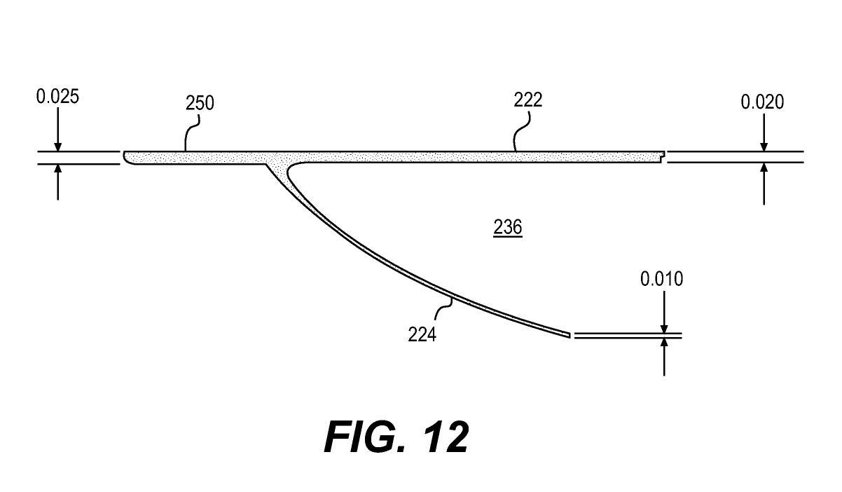

FIG. 12 is a partial broken away enlarged detailed cross-sectional view of the bandage bag 194, as indicated in FIG. 9. The dimensions of the various layers is indicated in inches. The thicker anterior wall 222 is laminated to the thinner posterior wall 224 to define the inferior edge 250 and pocket 236.

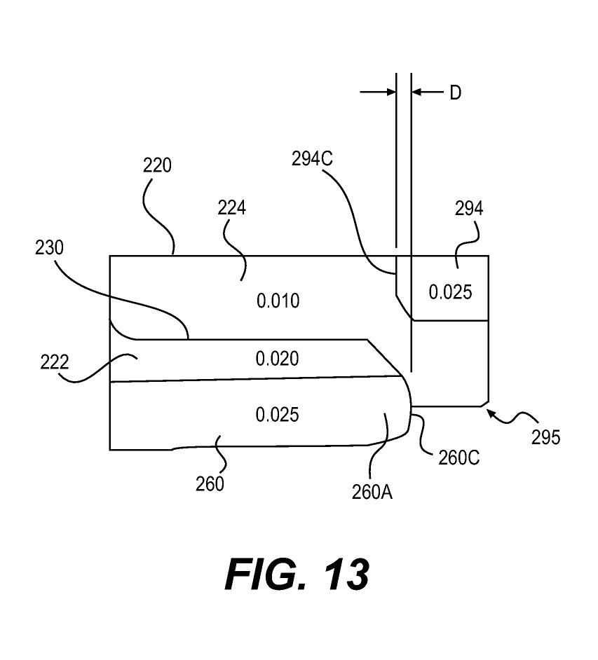

FIG. 13 is a partial broken away enlarged detailed view of one of the live hinges 295. There exists two live hinges 295, 295 defined between the ends 240A, 260A of the first lateral edge 240 and second lateral edge 260, respectively, and the U-shaped member 294. The two live hinges 295, 295 allow the enclosure bag 196 to be folded over the flap 292. The two live hinges 295, 295 together with the upper portion of the posterior wall 224 of the enclosure bag 196 define a combined live hinge between the flap 292 and enclosure bag 196. The axis of this combined live hinge is discontinuous with a hinge axis along a lower edge 294C of the U-shaped member 294 being offset from a hinge axis of the two live hinges 295, 295.

In the open position (i.e. flap open) of the bandage bag 194 shown in FIGS. 8-10, the U-shaped member 294 is at least partially nested (e.g. slightly nested) within the lateral edges 240, 260 of the enclosure bag 196. Specifically, the lower edge 294C of the U-shaped member 294 is slightly nested within the lateral edges 240, 260 of the enclosure bag 196. This is more clearly illustrated in FIG. 12 showing the lower edge 294C of the U-shaped member 294 being located slightly to the left a distance D relative to the edge 260B of end 260a of the lateral edge 260.

In the closed position (i.e. flap closed) of the bandage bag 194 shown in FIG. 11, the U-shaped member 294 is at least partially (e.g. substantially nested or fully nested) within the end lateral edges 240, 260 of the enclosure bag 196. Specifically, the U-shaped member 294 is fully nested within the lateral edges 240, 260 of the enclosure bag 196.

In use of the bandage bag 194 shown in FIG. 8, the U-shaped member is oriented with the wings 294A, 294A extending outwardly away from the enclosure bag 196 prior to be applied to the patient. The release backing 298 of the U-shaped member 294 of the bandage bag 194 is removed to expose the adhesive layer 296, and then the U-shaped member 294 is applied to the skin of the patient adjacent the catheter insertion site 170 (similar as in FIG. 3); however with the wings 294A, 294A of the U-shaped member 294 are positioned on either side of the catheter insertion site 170. Then the medical apparatus 180 (similar as in FIG. 3, tubing and port) is inserted into the bandage bag 10 through the bag opening 235. The release backing 290 of the edges 240, 260, 250 is removed, and then the enclosure bag 196 is bent along the live hinges 295, 295, and then folded over the U-shaped member 294 anchored to the skin of the patient. The enclosure bag 196 folded along the live hinges 295, 295 covers and protects the catheter insertion site 170. Thus, the live hinges 295, 295 are in a folded state when the enclosure bag 196 covers the catheter insertion site.

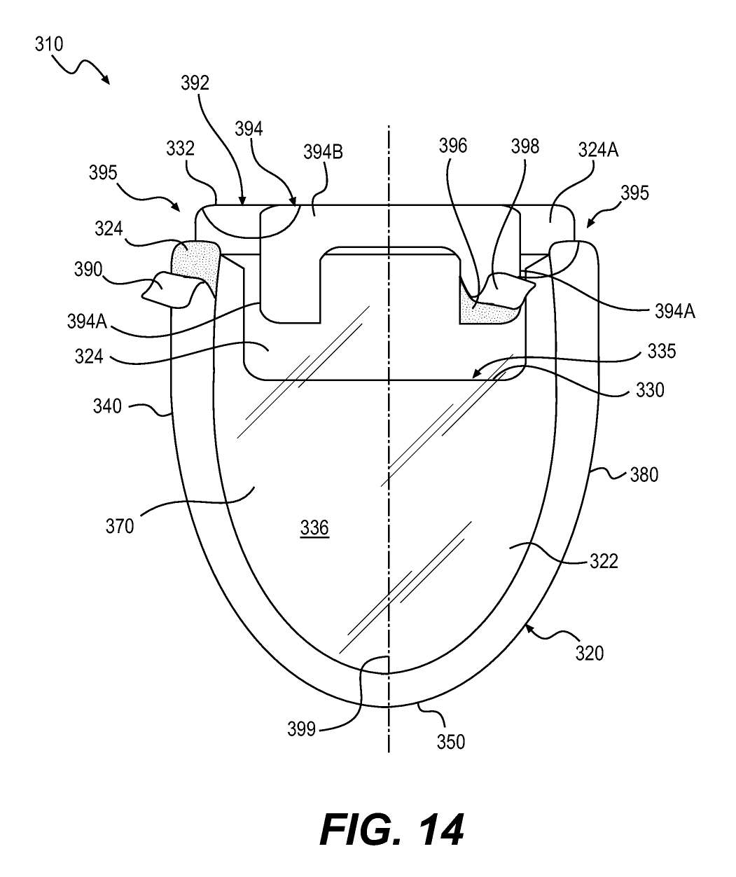

FIG. 14 is an anterior, or front, view of a further exemplary embodiment of the bandage bag 310, in a condition prior to being applied to a patient. The bandage bag 310 comprises an enclosure bag 320. The enclosure bag 320 includes a superior edge 330 on the anterior wall 322 of the enclosure bag 320 and a superior edge 332 on the posterior wall 324 of the enclosure bag 320 defining an opening 335 into a pocket 336. The enclosure bag 320 can be accessed through the opening 335 to place items, such as medical apparatus, inside the enclosure bag 320. The enclosure bag 320 has a first lateral edge 340, an inferior edge 350, and a second lateral edge 360. The anterior face 370 of the enclosure bag 320 is visible. A first adhesive layer 380 is disposed along the first lateral edge 340, inferior edge 350, and second lateral edge 360. Note that the adhesive 380 does not completely cover the anterior face 370, but only its edges. A removable backing 390 is shaped to cover the first adhesive layer 380 for ease and convenience in handling. The backing 390 is removed when the bandage bag 310 is used.

A flap 392 is provided at an upper portion of the enclosure bag 320. Specifically, the flap 392 is defined by an upper wall portion 324A of the posterior wall 324 of the enclosure bag 320. The flap 392 comprises a U-shaped member 394 comprising two spaced apart wings 394A, 394A connected together by a cross-member portion 394B. The wings 394A, 394A are oriented parallel to a longitudinal centerline 399 of the bandage bag 310.

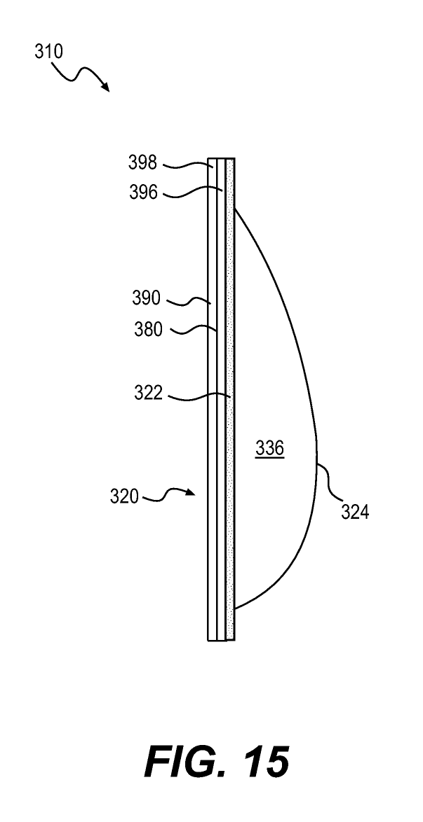

FIG. 15 is a side elevational view of the bandage bag 310 shown in FIG. 14. The posterior wall 324 of the enclosure bag 320 is curved outwardly to define the pocket 336 to place items, such as medical apparatus, inside the enclosure bag 320. The posterior side of the U-shaped member 394 is provided with an adhesive layer 396 and a removable backing 398.

In use of the bandage bag 310 shown in FIG. 14, the U-shaped member is initially oriented with the wings 394A, 394A extending inwardly and into the enclosure bag 320 prior to be applied to the patient. The release backing 398 of the U-shaped member 394 of the bandage bag 310 is removed to expose the adhesive layer 396, and then the flap 392 and U-shaped member 394 are folded outwardly and over, and then applied to the skin of the patient adjacent the catheter insertion site 170 (similar as in FIG. 3); however with the wings 394A, 394A of the U-shaped member 394 are positioned on either side of the catheter insertion site 170. Thus, in this embodiment (opposite to the embodiment shown in FIG. 8), the flap 392 is opened by lifting the U-shaped member 394 and folding the flap 392 prior to being applied to the skin. Thus, the flap 392 is folded to an open position prior to application

Then the medical apparatus 180 (similar as in FIG. 3, tubing and port) is inserted into the bandage bag 10 through the bag opening 335. The release backing 390 of the edges 340, 360, 350 is removed, and then the enclosure bag 320 is folded over the U-shaped member 394 anchored to the skin of the patient. The enclosure bag 320 bends along the live hinges 295, 295, and then covers and protects the catheter insertion site 170. Thus, the live hinges 295, 295 are in an unfolded state when the enclosure bag 320 covers the catheter insertion site.

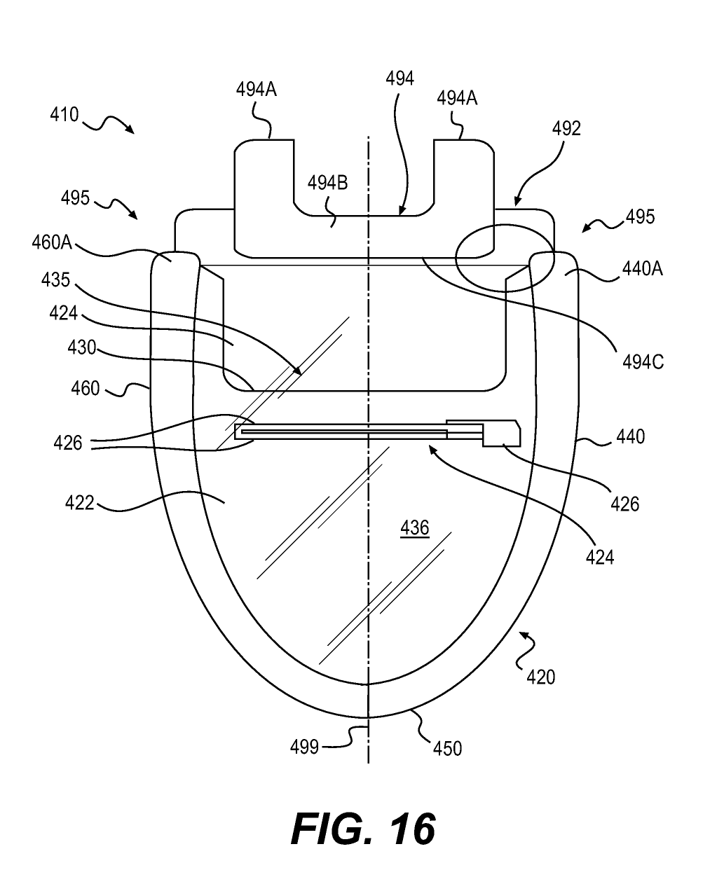

FIG. 16 is an anterior, or front, view of an even further exemplary embodiment of the bandage bag 410, in a condition prior to being applied to a patient. The bandage bag 410 comprises an enclosure bag 420. The enclosure bag 420 includes a superior edge 430 on the anterior wall 422 of the enclosure bag 420 and a superior edge 432 on the posterior wall 424 of the enclosure bag 420 defining an opening 435 into a pocket 436. The enclosure bag 420 can be accessed through the opening 435 to place items, such as medical apparatus, inside the enclosure bag 420. The enclosure bag 420 has a first lateral edge 440, an inferior edge 450, and a second lateral edge 460. The anterior face 470 of the enclosure bag 420 is visible. A first adhesive layer 480 is disposed along the first lateral edge 440, inferior edge 450, and second lateral edge 460. Note that the adhesive 480 does not completely cover the anterior face 470, but only its edges. A removable backing 490 is shaped to cover the first adhesive layer 480 for ease and convenience in handling. The backing 490 is removed when the bandage bag 410 is used.

A flap 492 is provided at an upper portion of the enclosure bag 420. Specifically, the flap 492 is defined by an upper wall portion 424A of the posterior wall 424 of the enclosure bag 420. The flap 492 comprises a U-shaped member 494 comprising two spaced apart wings 494A, 494A connected together by a cross-member portion 494B. The wings 494A, 494A are oriented parallel to a longitudinal centerline 499 of the bandage bag 410.

In this embodiment, the enclosure bag 320 is provided with additional closure, for example, a resealable closure 425. In the application of the resealable closure 425, the resealable closure 425 is configured to repeatedly open and close the enclosure bag 320 even after being closed by folding the enclosure bag 320 over the flap 492 and U-shaped member 494 (i.e. after being fully applied to the patient and closed). The resealable closure 425 provides an alternate access to the catheter and port stored in the enclosure bag without opening the bandage bag by detaching the enclosure bag 420 from the patient's skin and then unfolding the enclosure bag 420 to expose the primary opening 435 to gain access to the catheter and port.

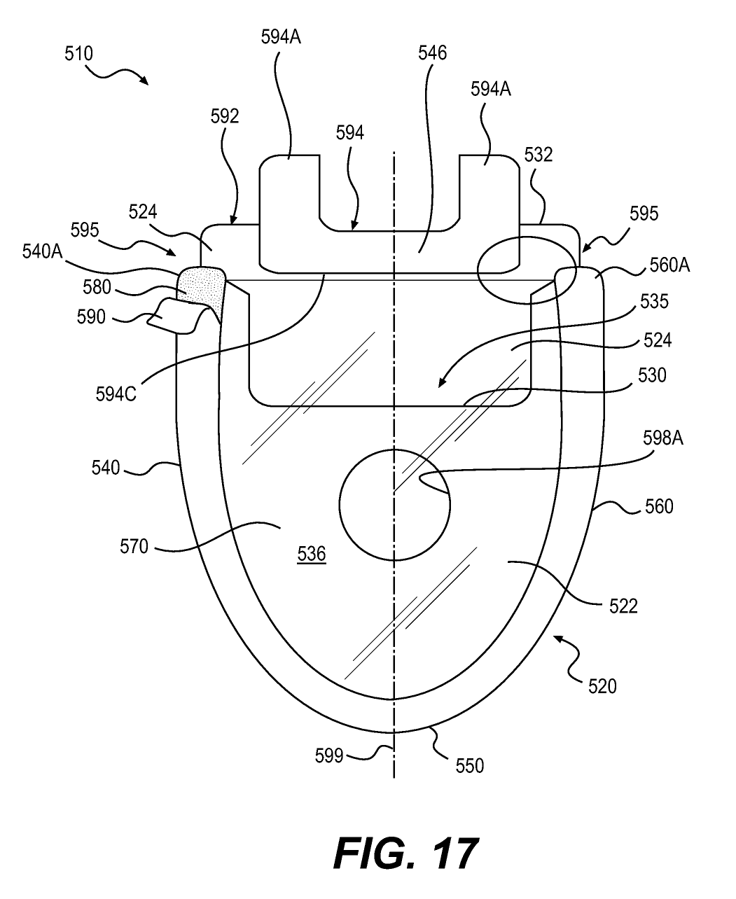

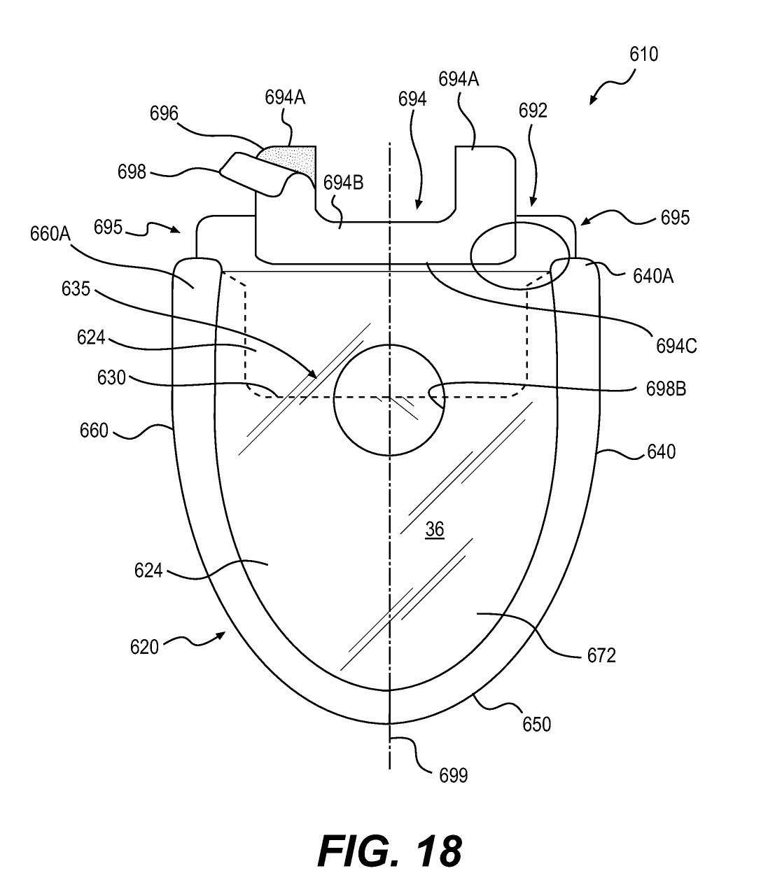

The resealable closure 425, for example, can be provided in the anterior wall 422, and comprise a pair of inter-connectable edges 426, 426 defining a closable opening and a slide configured to open the edges apart or close the edges together like a ZIPLOCK type seal/closure. The slide is at least accessible, for opening and closing the edges 426, 426, when the enclosure bag 420 is folded over the flap 492 and U-shaped member 494 when the bandage bag 410 is fully applied to the patient still allowing access to the catheter and port without unfolding the bandage bag 410. The resealable enclosure 425 can also be applied to the embodiments of the bandage bag 194 shown in FIG. 8, bandage bag 310 shown in FIGS. 14 and 15, bandage bag 410 shown in FIG. 17, and bandage bag 510 shown in FIG. 18.

Alternatively, the additional closure can be an opening defined by overlapping sections of the anterior wall 422, an open provided with a flap (e.g. resealable flap with semi-permanent adhesive), or a breakable or frangible line or pattern provided in the anterior wall 422.

The bandage bag can be a unitary structure by forming (e.g. molding) and/or laminating layers or membranes of material together. For example, the bandage bag can be made of a 40A silicone elastomeric material.

The bandage bag can be a relatively thin single layer or multiple layers of material. The resulting structure can be multiple zones, sections, portions, or areas of different or varying thickness. As shown in FIG. 13 the resulting construction provides multiple zones of different thickness that changes discretely (i.e. discontinuous) between adjacent or contiguous zones. Alternatively, the zones can be made to change thickness in a continuous matter (e.g. tapering between zones).

FIG. 17 is an anterior, or front, view of a further exemplary embodiment of the bandage bag 510, in a condition prior to being applied to a patient. The bandage bag 510 comprises an enclosure bag 520. The enclosure bag 520 includes a superior edge 530 on the anterior wall 522 of the enclosure bag 520 and a superior edge 532 on the posterior wall 524 of the enclosure bag 520 defining an opening 535 into a pocket 536. The enclosure bag 520 can be accessed through the opening 535 to place items, such as medical apparatus, inside the enclosure bag 520. The enclosure bag 520 has a first lateral edge 540, an inferior edge 550, and a second lateral edge 560. The anterior face 570 of the enclosure bag 196 is visible. A first adhesive layer 580 is disposed along the first lateral edge 540, inferior edge 550, and second lateral edge 560. Note that the adhesive 580 does not completely cover the anterior face 570, but only its edges. A removable backing 590 is shaped to cover the first adhesive layer 580 for ease and convenience in handling. The backing 590 is removed when the bandage bag 510 is used.

A flap 592 is provided at an upper portion of the enclosure bag 520. Specifically, the flap 592 is defined by an upper wall portion 524A of the posterior wall 524 of the enclosure bag 520. The flap 592 comprises a U-shaped member 594 comprising two spaced apart wings 594A, 594A connected together by a lower member portion 594B. The wings 594A, 594A are oriented parallel to a longitudinal centerline 599 of the bandage bag 510.

The bandage bag 510 is provided with a through hole 598A in the anterior wall 522 to allow tubing to extend through the hole 598A from inside the enclosure bag 520 to outside the bandage bag 510. For example, the tubing can extend through the hole 598A to the catheter insertion site or incision when the enclosure bag 520 is folded over the flap 592.

FIG. 18 is a posterior, or back, view of a further exemplary embodiment of the bandage bag 610, in a condition prior to being applied to a patient. The bandage bag 610 comprises an enclosure bag 620. The enclosure bag 620 includes a superior edge 630 on the anterior wall 622 of the enclosure bag 620 and a superior edge 632 on the posterior wall 624 of the enclosure bag 620 defining an opening 635 into a pocket 636. The enclosure bag 620 can be accessed through the opening 635 to place items, such as medical apparatus, inside the enclosure bag 620. The posterior face 672 of the enclosure bag 196 is visible. An adhesive layer 696 is disposed along the posterior side of the U-shaped member 694 for applying the bandage bag 610 to a patient.

The bandage bag 610 is provided with a through hole 698B in the posterior wall 624 to allow tubing to extend through the hole 698B from inside the enclosure bag 620 to outside the bandage bag 610. For example, the tubing extends through the hole 698B to a location above or outside the bandage bag.

The size and shape of the holes (i.e. hole 598A (FIG. 17) and/or hole 698B (FIG. 18) can be changed from that shown (e.g. large hole, smaller hole, different shaped hole, triangle-shaped hole, square-shaped hole, rectangular-shaped hole, custom-shaped hole). Further, the location of the holes can be changed or moved (e.g. anywhere on anterior side and/or posterior side of the enclosure bag). Further, multiple holes can be provide on one or both of the anterior side and/or posterior side of the enclosure bag (e.g. single hole on anterior side and single hole on posterior side of enclosure bag).

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

We claim:

1. A bandage bag, comprising:
a bag comprising an opening; and
a flap connected to the bag by a hinge.

2. A bandage bag, comprising:
a bag comprising an opening; and
a flap connected to the bag, the flap comprising a U-shaped member.

3. A bandage bag, comprising:
a thicker resilient layer of flexible material defining a perimeter of the bandage bag and a flap; and
a thinner resilient layer of flexible material defining a side of the bandage bag and a hinge between the bag and the flap, the thinner resilient layer laminated over the thicker layer.

4. The bandage bag according to claim 1, wherein the hinge is a live hinge.

5. The bandage bag according to claim 4, wherein the hinge is defined by a layer connecting the flap to the bag.

6. The bandage bag according to claim 5, wherein the flap comprises a U-shaped member.

7. The bandage bag according to claim 6, wherein the flap at least partially nests within a perimeter of the bag when the bandage bag is in an open position defining an interference type hinge between the flap and the bag.

8. The bandage bag according to claim 7, wherein the hinge is non-linear.

9. The bandage bag according to claim 6, wherein the U-shaped member defines a pair of wings.

10. The bandage bag according to claim 9, wherein the flap at least partially nests within a perimeter of the bag when the bandage bag is in an open position.

11. The bandage bag according to claim 7, wherein the flap at least partially nests within a perimeter of the bag when the bandage bag is in an open position.

12. The bandage bag according to claim 11, wherein the flap nests to a greater extend within a perimeter of the bag when the bandage bag is in an closed position verses in a closed position.

13. The bandage bag according to claim 1, wherein the flap comprises a U-shaped member.

14. The bandage bag according to claim 13, wherein the U-shaped member defines a pair of wings.

15. The bandage bag according to claim 14, wherein the wings are oriented parallel relative to a longitudinal centerline of the bandage bag.

16. The bandage bag according to claim 1, wherein one side of the bag comprises an outwardly curved bag wall.

17. The bandage bag according to claim 1, wherein the bag comprises a thicker resilient layer of flexible material defining a perimeter of the bandage bag, and a thinner resilient layer of flexible material defining a side of the bandage bag, the thinner resilient layer laminated over the thicker layer.

18. The bandage bag according to claim 1, wherein the flap is U-shaped, and a lower end of the U-shaped flap nests within a perimeter of the bag when the bandage bag is in an open position.

19. The bandage bag according to claim 1, wherein the flap is U-shaped, and an upper end of the U-shaped flap nests within a perimeter of the bag when the bandage bag is in an open position.

20. The bandage bag according to claim 1, wherein the bandage bag is provided with another opening.

21. The bandage bag according to claim 20, wherein the another opening is a resealable opening.

22. The bandage bag according to claim 21, wherein the resealable opening is a ridge and channel type resealable closure.
